(19) United States
(12) Patent Application Publication
(10) Pub. No.: US 2006/0188895 A1
(43) Pub. Date: Aug. 24, 2006

(54) Rapid prognostic assay for malignancies treated with epidermal growth factor receptor

(75) Inventors: Piotr Kulesza, Baltimore, MD (US); Antonio Jimeno, Baltimore, MD (US); Manuel M. Hidalgo, Baltimore, MD (US); Douglas P. Clark, Baltimore, MD (US); Erik A. Kincaid, Baltimore, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 11/217,657

(22) Filed: Aug. 31, 2005

Related U.S. Application Data
(60) Provisional application No. 60/605,929, filed on Aug. 31, 2004.

(51) Int. Cl. C12Q 1/68

(52) U.S. Cl. 435/6

(57) ABSTRACT

A molecular assay for determining the sensitivity or resistance of malignancies to chemotherapy prior to initiation of chemotherapy and which also allows for monitoring the therapeutic effects of the chemotherapy during treatment. The molecular assay measures tumor response to therapy with EGFR modulators and utilizes tumor mRNA as a starting material and a quantitative measurement of c-fos expression as an analytical endpoint.

### CROSS-REFERENCE TO RELATED APPLICATIONS

This application claims the benefit and priority of U.S. provisional patent application having Ser. No. 60/605,929 filed Aug. 31, 2004 which is herein incorporated by reference.

### FIELD OF INVENTION

The invention is generally directed to a diagnostic assay which evaluates the sensitivity or resistance of malignancies to targeted chemotherapy prior to initiation of chemotherapy and which also allows for monitoring the therapeutic effects of the chemotherapy on the malignancies. More particularly, the invention is directed to a molecular assay for measuring tumor response to therapy with Epidermal Growth factor Receptor (EGFR) modulators which utilizes tumor mRNA as a starting material and a quantitative measurement of c-fos expression as an analytical endpoint.

### BACKGROUND OF THE INVENTION

The control of cell growth relating to cancer research is of particular importance because uninhibited growth of cells may result in tumor formation. Growth factor proteins are signaling molecules that bind to specific receptor proteins on the surface of cells to regulate the many genes involved in cell growth. A sequence of reactions changing the function of the cell is initiated after binding of the growth factor proteins to the specific receptor proteins.

Cell or tissue response to specific growth factors is determined by the growth factor receptors the cell or tissue possesses and the intracellular reactions initiated when any one growth factor binds to its receptor. One example of a receptor-growth factor system is the stimulation of cell proliferation by binding epidermal growth factor (EGF) to epidermal receptor proteins on the surface of epidermal cells and other cells. The epidermal growth factor receptor (EGFR) is a single polypeptide chain that spans the plasma membrane. The EGFR has an extracellular domain, a single alpha-helix transmembrane domain, and an intracellular domain with tyrosine kinase (TK) activity. Ligand or combination of ligand binding induces EGFR homodimerization and heterodimerization with other HER proteins, activation of TK activity, and autophosphorilation. EGFR signaling ultimately increases proliferation, angiogenesis, metastasis, and decreases apoptosis. Studies on gefitinib, the generic name of a drug used to treat several types of cancer that is manufactured under the brand name Iressa by AstraZeneca located in Wilmington, Pa., and erlotinib, the generic name for a drug approved for treating non-small cell lung cancer, and being tested for other types of cancer, that is manufactured under the brand name Tarceva by Genentech located in San Francisco, Calif., which are both quinazoline derivatives that reversibly inhibit the TK activity of EGFR show both in vitro and in vivo activity in human cancer cell lines.

Despite the ubiquitous expression of the EGFR, and the large number of patients treated in the context of clinical trials with EGFR-targeted agents, the factors determining and predicting their efficacy are largely unknown. Initial reports have suggested that the presence of acquired mutations in the catalytic domain of the egfr gene increase sensitivity to anti-EGFR small-molecule modulators in non small cell lung cancer, and hypothesized that these mutations are the basis for success of therapy with EGFR modulators. However, subsequent studies in lung cancer, and numerous studies in other cancers have not substantiated this hypothesis. Therefore, factors predicting sensitivity to EGFR blockade are unknown, and new strategies are being sought after to individualize cancer therapy.

The invention described herein offersadvantages, as it exploits specifically the functional consequences of EGFR inhibition, irrespective of the many possible etiologic causes of cancer.

A component in the response to proliferative signals is the rapid, transient transcriptional activation of immediate early genes, such as the c-fos proto-oncogene. C-fos expression is regulated at multiple levels by intracellular signalling events, which makes it a useful paradigm to identify and characterize factors that affect cancer cell growth. C-fos is a robust marker of proliferation, and we have utilized it as a distal marker to assess EGFR activation, and anti-EGFR therapy. The present invention utilizes this marker to develop a molecular response assay to predict sensitivity to EGFR blockade using an ex vivo approach.

### SUMMARY

Described herein are diagnostic assays which evaluates the sensitivity or resistance of malignancies to targeted chemotherapy prior to initiation of the chemotherapy and monitors the therapeutic effects of the chemotherapy on the malignancies.

One exemplary embodiment of the invention includes a method for determining susceptibility of tumor cells to a chemotherapeutic agent prior to initiation of chemotherapy which includes the steps of obtaining tumor cells, incubating first, second, third and fourth equal volumes of the tumor cells with media, introducing a chemotherapeutic agent to the third and fourth equal volumes of tumor cells contained in the media for a predetermined amount of time, exposing EGFR ligand or combinaiton of ligand to the second and third equal volumes of tumor cells contained in the media, quantifying a level of c-fos mRNA in the first, second, third and fourth equal volumes of tumor cells wherein the levels of c-fos mRNA are expressed in ng/mL, adjusting the levels of c-fos mRNA for comparison by dividing the c-fos mRNA levels in the first, second, third and fourth equal volumes of tumor cells by the c-fos mRNA level in the first equal volume of tumor cells, and comparing the adjusted levels of c-fos mRNA for the second equal volume of tumor cells to the adjusted level of c-fos mRNA for the third equal volume of tumor cells to determine whether c-fos mRNA expression has been suppressed. It will be understood by those skilled in the art that the EGFR ligand or combinaiton of ligand may be a product of any ErbB receptor encoded by any gene from the erbB gene family, and any homo- and heterodimers that these molecules are known to form.

The chemotherapeutic agent in the above described exemplary embodiment may be an EGFR modulator and obtaining tumor cells may include conducting fine needle aspiration. In addition, introducing a chemotherapeutic agent may include introducing the chemotherapeutic agent to the third and fourth equal volumes of tumor cells for between about 1 minute and about twelve hours prior to exposing EGFR ligand or combination of ligand to the second and third equal volumes of tumor cells.

The step of quantifying a level of c-fos mRNA in the first, second, third and fourth equal volumes of tumor cells described above may be performed at predetermined time intervals at any of the times between ten minutes and four hours after the step of exposing EGFR ligand or combination of ligand to the second and third equal volumes of tumor cells. Further, quantifying the the level of c-fos mRNA in the first, second, third and fourth equal volumes of tumor cells may be determined using quantitative real time polymerase chain reaction (Q-PCR) following reverse transcription of the mRNA into cDNA.

In one aspect, methods for determining susceptibility of tumor cells in response to a chemotherapeutic agent prior to initiation of chemotherapy comprising are provided. The method includes, incubating at least a first and a second volume of tumor cells with media; introducing a chemotherapeutic agent to the first volume of tumor cells in media for a predetermined amount of time; exposing Epidermal Growth Factor Receptor (EGFR) ligand or combinaiton of ligand to the second volume of tumor cells in media; quantifying a level of c-fos mRNA in the first and second volumes of tumor cells; adjusting the levels of c-fos mRNA for comparison; and comparing the adjusted levels of c-fos mRNA to determine whether c-fos mRNA expression has been suppressed.

“Determining susceptibility,” as used herein refers to the ascertainment of patient condition as it relates to subjects disease, e.g., malignant disease. It also relates to treatment, diagnosis of patients.

Media, as used herein, includes, cellular media, serum free media and growth media.

The invention is also directed to a method for monitoring the therapeutic effects of chemotherapeutic agents which includes the steps of utilizing tumor mRNA as a starting material and measuring c-fos expression as an analytical endpoint. The step of utilizing tumor mRNA as a starting material may include the step of directly extracting mRNA from tumor cells at predetermined intervals during treatment with a chemotherapeutic agent such as an EGFR modulator. Like the step of obtaining tumor cells in the method for determining susceptibility of tumor cells to a chemotherapeutic agent prior to initiation of chemotherapy, the step of directly extracting mRNA from tumor cells may be carried out by fine needle aspiration. The step of measuring c-fos expression may be determined using real time Q-PCR following reverse transcription of the mRNA into cDNA.

Both methods described above utilize a molecular assay which uses tumor mRNA as a starting material and a quantitative measurement of c-fos expression as an analytical endpoint. The method for determining susceptibility of tumor cells to a chemotherapeutic agent uses non-treated tumor mRNA and the method for monitoring the therapeutic effects of chemotherapeutic agents uses tumor mRNA that has been treated with a chemotherapeutic agent such as an EGFR modulator.

The diagnostic molecular assay of the invention can be performed with minimal morbidities and discomfort and significantly shortens the time frame for assessing the responsiveness of tumor cells to anti-cancer therapies.

### BRIEF DESCRIPTION OF THE DRAWINGS

FIG. 1 is a table showing that c-fos expression (expressed as fold increase normalized to treatment with media) is stimulated with a short exposure to EGF and also suppressed by a 1 hour EGFR inhibition with gefitinib 1 μM or gefitinib 10 μM (provided under the brand name Iressa by AstraZeneca in the presence or absence of EGF) in some cell lines but not in others. The “ex vivo” assay, done on cell lines in vitro, predicted the in vivo sensitivity to therapy with EGFR modulator.

FIG. 2 is a graph showing the data set out in FIG. 1 where EGF+gefitinib is EGFR modulator with gefitinib 10 μM.

FIG. 3 is a graph showing that growth inhibition as assessed by MTT after 72 hours correlates with c-fos downregulation after both 10 μM gefitinib and 10 μM erlotinib treatment.

FIG. 4 is a graph showing that the modulatory effect on c-fos was modestly increased with dose, and was maintained up to 5 days.

FIG. 5 is a set of graphs showing In vivo growth of 5 cell lines xenografted in nude mice treated with gefitinib provided under the brand name Iressa by AstraZeneca (Δ) or erlotinib provided under the brand name Tarceva by OSI Pharmaceuticals (▪) and evolution of c-fos levels. Left graphs show tumor growth plots. Right graphs show relative c-fos levels as assessed by RT-Q-PCR. Growth values are expressed as percentage relative to baseline ±SD (n=12 tumors per group). Tumor c-fos levels were assessed by means of sequential fine needle aspirations of 7-8 tumors per treatment group at baseline and after 14 days of therapy, and each day 14 value was normalized in percentage to its baseline value, allowing a paired intra-tumor comparison.

FIG. 6 is a graph showing ex vivo assessment of material obtained by fine needle aspiration from 7 CAL27 tumors from different mice. As in the in vitro culture, EGF stimulation induced c-fos upregulation, and erlotinib provided under the brand name Tarceva by OSI Pharmaceuticals was able to abrogate this.

FIG. 7 is a graph showing the relationship between changes in c-fos and the proliferation index Ki67 in paired tumor samples of patients treated with gefitinib provided under the brand name Iressa by AstraZeneca (500 mg/day). In both patients where a significant (<50% from baseline) decrease of Ki67 was documented the post-treatment levels of c-fos were lower compared with baseline levels; however, patient #3 presented an increase in c-fos whilst having a 45% reduction of Ki67.

### DETAILED DESCRIPTION

In general, provided herein aremolecular assays capable of both evaluating the sensitivity or resistance of a patient's malignancy to a chemotherapeutic agent, and in particular an EGFR modulator, prior to initiation of therapy and monitoring the therapy effects during treatment. The diagnostic assay directs therapy and determines prognosis of patients treated with targeted anti-cancer therapy. The assay is based on fine needle aspiration of any neoplastic lesion and processing the aspirated material for mRNA analysis. Depending on the particular pharmaceutical agent used, the assay allows for determination of sensitivity of the lesion to treatment, effectiveness of specific pathway blockade, and monitoring of therapy effects at the molecular level. The assay can be performed with minimal morbidities and discomfort, and can be used for drug sensitivity assessment, dosing regimen, therapy effect measurement, and prognostication.

EGFR modulators, as used herein, include for example, EGFR inhibitors and activators. Exemplary modulators include small molecules (e.g. erlotinib, gefitinib, or lapatanib), antibodies (e.g., Herceptin).

Combination of ligands, includes, for example, one or more of EGF, TGF-a, and Heregulin.

Since the assay allows for determination of susceptibility of malignant lesions to targeted chemotherapy prior to initiation of chemotherapy, chemotherapy treatment can be specifically tailored for each individual patient. The ability to determine the susceptibility of malignant lesions to targeted chemotherapy avoids the long wait time (often several months) before response to therapy can be assessed. In cases where there is resistance to therapy, there can be severe and/or irreversible deterioration of the patient's clinical condition during the months of treatment thereby precluding any possibility of further alternative treatment.

The inventive assay allows for the specific measurement of pathway inhibition by a pharmaceutical agent, such as and EGFR modulator, which has not previously been available. The inventive assay also allows for monitoring the effect of the drug on any neoplastic lesion for either immediate prognostication, or prognostication within weeks of initiation of therapy. In the past, this was measured by radiographic regression of lesions in a much longer time frame (e.g., over months).

The assay requires the procurement of lesion tissue. Although fine needle aspiration to procure lesion tissue may be preferred in most instances, it should be understood that any and all other ways known in the art for procuring lesion tissue are contemplated by the invention. The procedure for procuring lesion tissue is quick and can be done in an outpatient setting. Sample processing can be performed by any standard of care cytopathology laboratory. The end-point analyses require a molecular pathology laboratory for mRNA analysis which includes a standard tissue culture facility, commercially available RNA extraction kits, cDNA processing, and a quantitative PCR machine.

In this submission we use the term “EGFR” to indicate erbB gene famility products. It will be understood by those skilled in the art that the EGFR may be a product of any erbB receptor encoded by any gene from the erbB gene family, and any homo- and heterodimers that these molecules are known to form. While erbB-1 product is the main receptor, and we detected its expression in our studies, there is reason to believe that the cell lines and tumors we used also express other erbB gene familiy members (since all tumors we used are derived from epithelial malignancies). Lastly, the EGFR ligand or combination of ligand we used binds to almost all of the known EGFR receptor forms, and therefore our assay measures the effects exerted by those proteins.

Materials and Methods

Drugs

Gefitinib was provided by AstraZeneca located in Wilmington, Del. under the brand name Iressa. Erlotinib was provided by OSI Pharmaceuticals located in Melville, N.Y. under the brand name Tarceva.

Cell Lines and in Vitro Culture Conditions

Five cell lines were used: A431, Cal27, HN11, HuCCT1, and Hep2. HN11 was a gift from Dr David Sidranski's laboratory at Johns Hopkins University (Baltimore, Md.), and A431, Cal27, HuCCT1, and Hep2 were obtained from the American Tissue Culture Collection (Manassas, Va.). A431 is a squamous cell carcinoma, Cal27, HN11 and Hep2 are derived from head and neck squamous carcinomas, and HuCCT1 is a cholangiocarcinoma. The cell lines were grown in 6-well plates with DMEM supplemented with 10% fetal bovine serum (FBS) and penicillin/streptomycin. After overnight serum-starvation cells were treated either with growth media (GM), GM plus human epidermal growth factor 100 ng/mL (EGF; Sigma), GM plus EGF and gefitinib, or GM plus gefitinib. The cells were incubated for 1 hour, media was aspirated, and RNA collected by direct in-well lysis with 0.5 mL of RLT (Qiagen). A subsequent experiment involved treatment of the cells during 24 hours with GM, GM plus gefitinib or GM plus erlotinib. IN VITRO GROWTH INHIBITION STUDIES

In vitro drug sensitivity to concentrations of gefitinib and erlotinib ranging from 0-10 μM was assessed by MTT (manufactured by Sigma located in St Louis, Mo.) following the manufacturer's instructions. Briefly, cells were seeded at 5×10³ cells/well in 96-well plates and grown for 24 hours before treatment with exponentialy increasing concentrations of gefitinib or erlotinib in the presence of 10% FBS.

In Vivo Growth Inhibition Studies

Five groups of 14-16 six-week-old female athymic nude mice (Harlan, Ind., US) were used. 1.5-5×10⁶ A431, CAL27, HN11, HuCCT1, and Hep2 cells were injected subcutaneously in each flank. Tumors were grown to a size of 0.2 cm³, and mice were stratified by tumor volume into different groups (5-6 mice [10-12 tumors] per group) that were treated with vehicle, gefitinib 100 mgr/kg intraperitoneally (IP) once a day for 14 days (A431 and HuCCT1), or erlotinib 50 mgr/kg IP once a day for 14 days (CAL27, HN11, and Hep2).

Fine Needle Aspiration (FNA)

FNAs on mice were performed according to standard cytopathologic practice under inhaled general anesthesia (isofluorane) using 10 cc syringes and 25-gauge needles. During each FNA procedure the first pass was smeared onto glass slides and used for morphologic analysis, (DiffQuik™ and Papanicoloau), and the second and third passes for RNA extraction. Eighteen A431 tumors, fourteen CAL27 tumors, eighteen HN11 tumors, fourteen HuCCT1 tumors, and sixteen Hep2 tumors were tested. FNAs were performed at baseline and after 14 days of therapy for each of the tumors. Tumor biopsies on patients were performed following an ultrasonographic-guided, FNA-assisted methodology, with on-site cytopathologic assessment of tissue adequacy at baseline and after 28 days of therapy.

Ex Vivo Molecular Assay

Material collected by two FNA passes on seven CAL27 xenograft tumors at baseline was aliquoted in growth media, and treated in tissue culture by short (30 to 60 minutes) exposure to GM, GM plus EGF 100 ng/mL, GM plus EGF and erlotinib, or GM plus erlotinib.

Patients and Study Design

Materials were collected from five consecutive patients treated in a clinical trial aiming to determine the biologic effects of gefitinib. Patients were required to be 18 years of age or older, and have histologically documented metastatic or inoperable malignancy amenable to sequential biopsies, and for which there was no known curative or standard palliative regimen (or failure of such regimens have occurred). Gefitinib was administered at a dose of 500 mg daily on an uninterrupted basis. The scientific review board of our institution granted protocol approval and patients were required to provide written informed consent prior to enrollment into the study.

RNA Extraction

In vitro RNA extraction was performed on non-confluent cells after treatment. Wells were washed with PBS and RLT lysis buffer was added. Two passes from the FNA were put in lysis buffer (Mini Rneasy manufactured by Qiagen) loaded onto a column, washed and eluted into 50 μl TE pH8. Total RNA was extracted using the RNeasy™ Mini Kit (Qiagen, Valencia, Calif.). RNA was transcribed into cDNA by reverse transcription by priming with random hexamers (M-MLTV manufactured by Promega located in Madison, Wis.). The excess hexamers were removed using a column-based clean-up kit (Qiagen).

Quantitative Real-Time RT-PCR Analysis

Quantitative PCR was performed on an MX3000p thermal cycler (Stratagene) using SYBR green dye method to track the progress of the reactions with ROX dye added as reference. Beta-Globin DNA specific primers were used to test DNA contamination for each sample type. Three housekeeping genes (HPRT, UBC and SDHA) were run in parallel with test genes. The amount of change in the target gene between the control and experimental conditions was found by comparing the threshold cycle (Ct) of the target gene to the geometric mean of the threshold cycles of the housekeeping genes. The geometric mean of the Cts of each of the housekeeping genes, and a change in threshold cycle (delta Ct), between conditions were calculated (dCt_{Houskeeping}=(Ct_{HPRT}*Ct_{UBC}*Ct_{SDHA})_{control}−(Ct_{HPRT}*Ct_{UBC}*Ct_{SDHA})ₑₓₚ). The change in threshold cycle for the target gene was calculated directly from Ct under each condition (dCt_{target}=(Ct_{target})_{control}−(Ct_{target})ₑₓₚ). The efficiency of the housekeeping genes raised to their dCt divided by the efficiency of the target gene raised to its dCt gave a ratio between the control and experimental conditions normalized to the housekeeping genes (Ratio=E_{Target}^{dCtTarget}/E_{Housekeeping}^{dctHousekeeping}, where E=Primer efficiency, and Ct=Threshold cycle).

Immunohistochemical Analysis

Core biopsies from patients were processed using standard procedures (formalin-fixed, and paraffin embedded). Five-micron sections were used for Ki67 staining that was performed following the manufacturer's instructions (M7187 manufactured by DAKO located in Carpinteria, Calif.).

c-fos increases selectively after exposure to EGF in TKI-sensitive cell lines

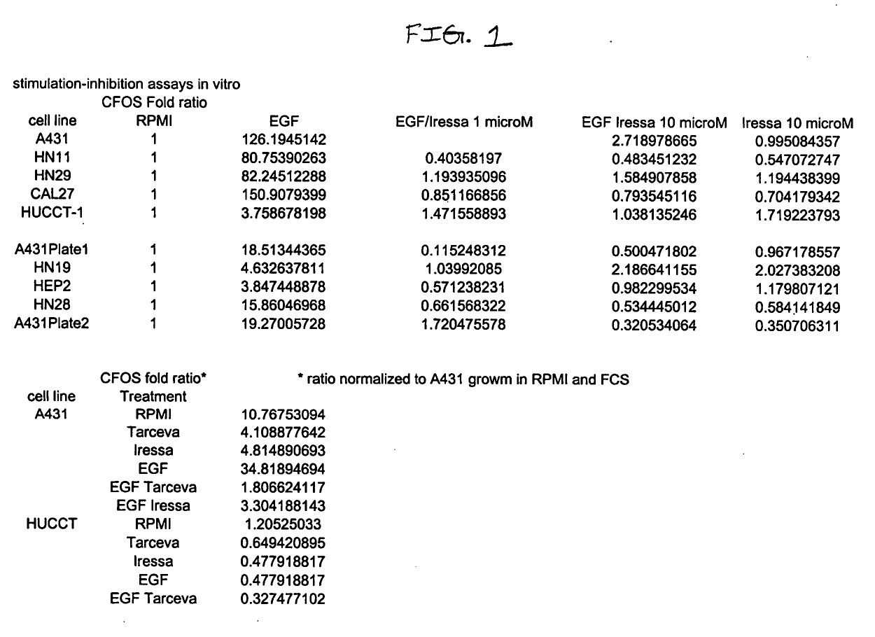

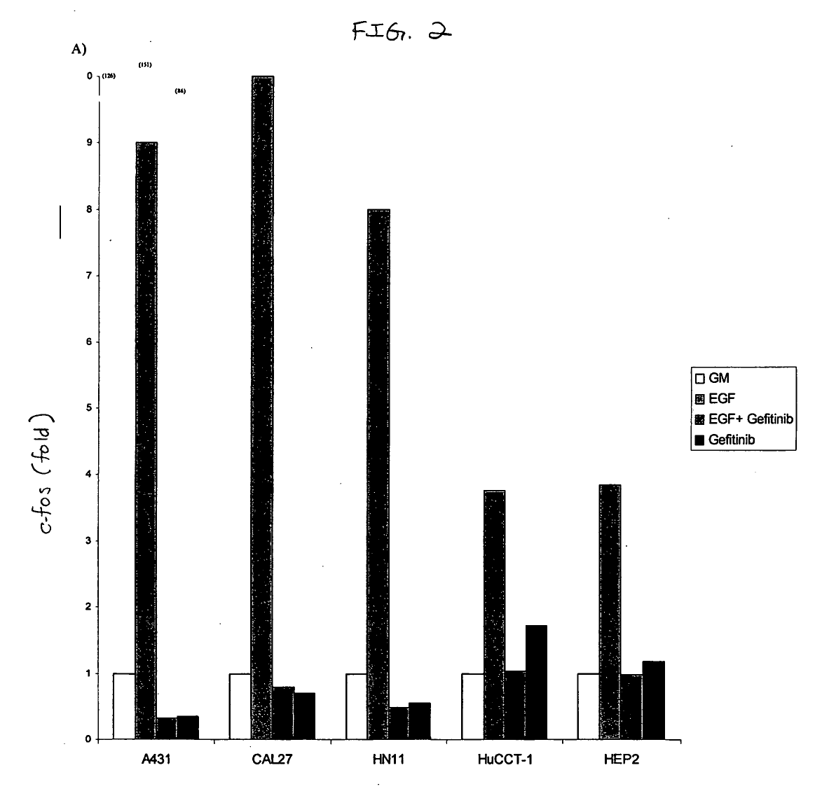

After a brief exposure to EGF, A431, CAL27 and HN11 showed markedly elevated levels of c-fos mRNA (126, 151 and 86-fold, respectively); these EGF-induced increments were abrogated when gefitinib was subsequently added for a short, 1-hour exposure at 10 μM (see FIGS. 1 and 2). Gefitinib alone also decreased c-fos levels in these cells. In contrast, HuCCT1 and Hep2 showed modest (3.6 and 4.1-fold) c-fos increases upon exposure to EGF; gefitinib alone had no effect on c-fos levels.

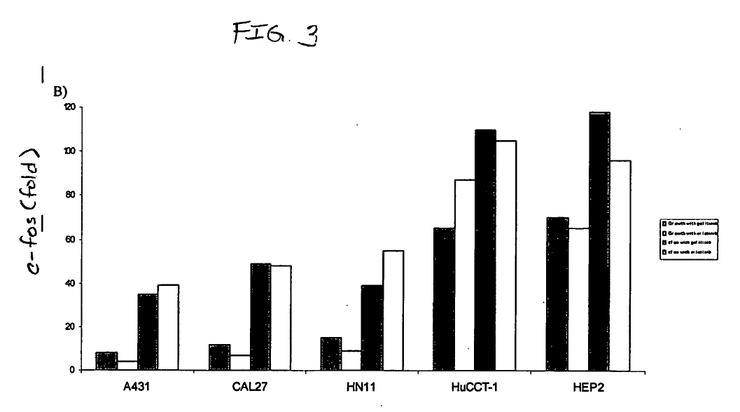

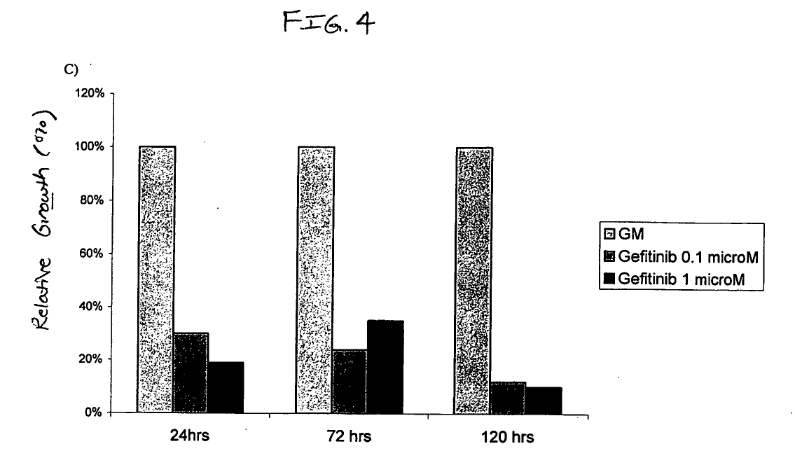

The effect of a longer exposure to both gefitinib and erlotinib was then assessed with regards to cell growth and c-fos dynamics. Cell lines with EGF-inducible c-fos upregulation showed high (and parallel) in vitro sensitivity to both agents (see FIG. 3); a longer (24-hour) exposure to EGFR modulators caused a lasting c-fos downregulation. HuCCT1 and Hep2 showed high level (IC50 >10 μM) resistance to inhibition, and c-fos levels minimally increased with time compared to baseline. In an experiment in A431 cells to examine time and dose-dependency, gefitinib at 0.1 and 1 μM decreased c-fos effectively at 24, 48 and 120 hours (see FIG. 4); dose-dependency was seen at 24 hours, but not at 48 or 120 hours.

The in vitro sensitivity c-fos assay predicts reponse to therapy in murime model

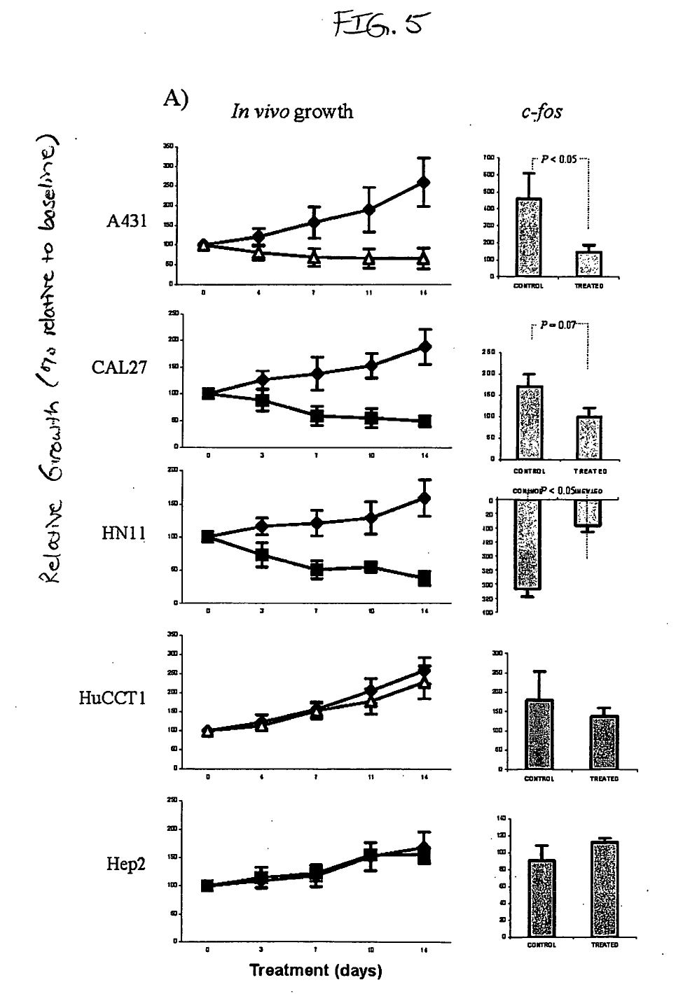

Four other cell lines (A431, HN11, HEP2 and HuCCT1-1 were also tested in the above manner in an in-vitro setting. The levels of c-fos in FIG. 1 correlate to response to therapy in a xenograft model as shown in FIG. 5. That is, the in-vitro assay predicted in vivo sensitivity to EGFR inhibition: A431 and HN11 tumors are sensitive, Hep2 and HuCCT-1 are resistant.

Ex Vivo Molecular Assay

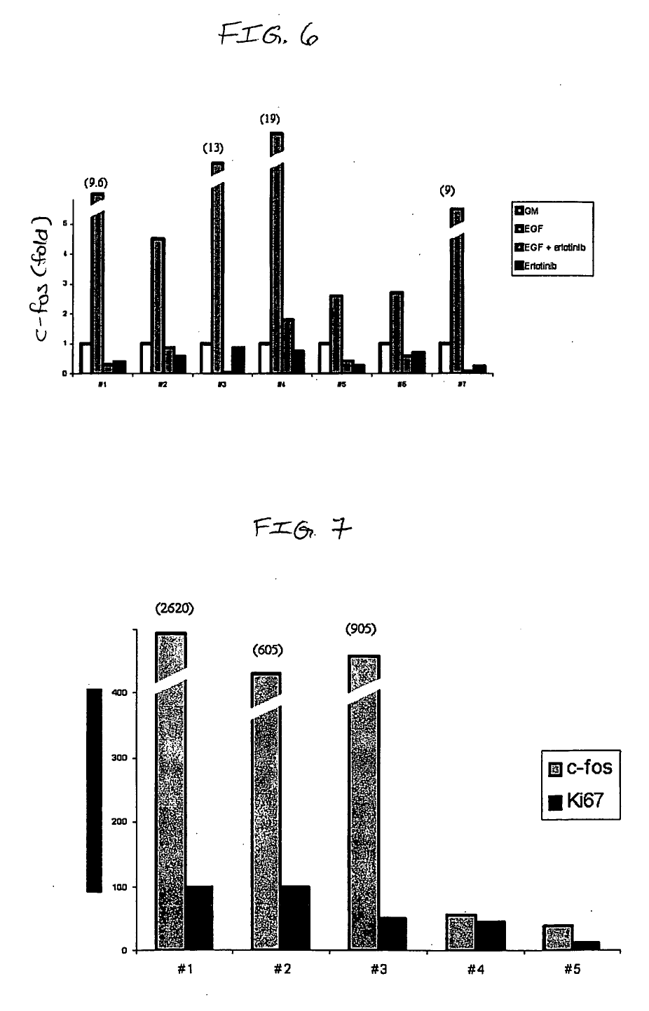

The ex vivo results on FNA-acquired tumor material from seven CAL27 xenograft tumors paralleled those obtained in cell culture, with 3 to 19-fold c-fos upregulation upon EGF stimulation and abrogation of this response with erlotinib; this experiment showed that the inhibition is achievable by either small-molecule modulator, as the in vitro was done with gefitinib (see FIG. 6).

In vivo tumor growth modulation and c-fos changes in response to gefitinib and erlotinib

To confirm the molecular events described before, and to determine the effect of these drugs in a model closer to a clinical context, A431, CAL27, HN11, HuCCT1, and Hep2 in vivo models were generated (see FIG. 5). Gefitinib or erlotinib induced growth arrest in A431, CAL27 and HN11 tumors. In A431, CAL27 and HN11 the average c-fos at 14 days is 4.8, 1.8 and 3.2-fold compared with baseline in control mice, respectively (baseline vs. day 14 for control mice, P<0.05 in all three). Gefitinib or erlotinib were able to significantly abrogate the progressive increase in c-fos expression observed in the control mice with time (day 14 control vs. day 14 treated, P<0.05 in A431 and HN11, P=0.07 in CAL27). In HuCCT1 and Hep2 xenografts no response was observed after treatment, c-fos levels did not increase significantly with time, and they were unchanged by EGFR modulators.

c-fos dynamics correlate with Ki67 proliferation index evolution in patient tumor samples

The paired tumor material from five unselected, consecutive patients was used for this analysis. The patients (henceforth numbered #1-5) presented colorectal (#1 and #3), non-small cell lung (#2), breast (#4) and neuroendocrine (metastatic carcinoid) (#5) carcinomas, and received 2, 4, 2, 2, and 5+ 1-month cycles of gefitinib. No objective, confirmed responses were documented, and hence for the purpose of correlations with c-fos evolution, the Ki67 proliferation index was selected as the efficacy endpoint variable. Three patients showed markedly (26, 6.1, and 9.1-fold) increases in c-fos after 28 days, and in two patients c-fos decreased (to 56% and 37% of baseline values) (see FIG. 7). Ki67 index was not influenced by therapy in two patients, whereas it decreased to 52%, 42% and 10% of baseline values in patients #3-5, respectively. In both patients where a significant decrease of Ki67 was documented (<50% from baseline) the post-treatment levels of c-fos were lower compared with baseline levels.

There is an increasing interest in examining determinants of response to anticancer agents as tools to prospectively tailor therapy to individuals more likely to benefit from the drugs. It is a strategy that is intuitive and appealing from both a clinical and a financial standpoint. This scrutiny is especially evident in the case of novel targeted therapies, and proof-of-principle pilot analyses are increasingly being embedded into clinical protocols. This invention provides assessment of c-fos dynamics to predict the activity of EGFR TKI and development of this marker as an ex vivo tool that can be incorporated in clinical studies and in devising individual chemotherapy treatment plans.

C-fos levels increased after EGF stimulation and were inhibited by anti-EGFR agents in vitro in cell lines that are naturally sensitive to EGFR modulators, but not in those intrinsically resistant. c-fos levels increase and correlate with tumor growth in untreated control tumors corresponding to EGFR TKI-sensitive cell lines, and c-fos mRNA dynamics correlates with tumor response to gefitinib and erlotinib in a xenograft model in both sensitive and resistant cell lines. In the current experiments the assessment of a proximal endpoint (EGFR inhibition) was less specific in prognosticating outcome than evaluating a distal endpoint (c-fos downregulation). An aspect of these embodiments is the increase in c-fos expression with time seen in the untreated mice; this may relate to EGFR-dependence but may also have a component of tumor growth-driven stimulation. Interestingly, other studies have shown c-fos levels to be similar between normal and tumor tissue in HNSCC patient samples, but significantly higher in tumor tissue in esophageal cancer patients.

C-fos was sequentially assessed on patient-derived material that was preliminary tested with a series of five unselected patients receiving gefitinib and undergoing pre- and post-therapy FNA-guided tumor biopsies. Unfortunately none of the patients responded to therapy, and we compared c-fos dynamics to Ki67 proliferation index variability instead; interestingly, in patients where a significant (<50% from baseline) decrease of Ki67 was documented a decrease in the post-treatment levels of c-fos was observed compared with baseline. We have previously shown that Ki67 proliferation index is a good surrogate efficacy marker in anti-EGFR therapy.

Fine needle aspiration (FNA) has demonstrated to be a robust and safe method to acquire tumor material in sufficient quantities to assess pharmacodynamic endpoints in a serial manner. In addition, preliminary evidence is provided suggesting that this methodology can be efficiently used in procuring tissue to reproduce in vitro conditions and develop an ex vivo molecular sensitivity and resistance assay. This approach has classically drawn considerable interest and the outcome and ultimate significance of a number of these studies has been the subject of recent reviews. Most studies analyzed whether cells derived from a sample of viable tumor tissue show a response when exposed to selected therapeutic agents under in vitro conditions. In the main, cloning and proliferation assays are used for this purpose, which suffer from many disadvantages such as setup complexities, and the necessity for some growth of lesional tissue under in-vitro conditions. Consequently, lack of reproducibility has prevented these appealing strategies from being incorporated to the clinical practice. However, if a robust correlation can be established between a given pharmacodynamic effect and outcome in preclinical models and pilot clinical studies, molecular testing has several advantages when compared with proliferation assessment: it requires a lower amount of tumor cells, active ex vivo proliferation is not a requirement (although cells have to maintain viability), and short-term exposure as opposed to long-term treatment is sufficient to elicit an assessable response.

Evaluating c-fos dynamic behavior in accordance with the invention is useful early in the course of treatment, before clinical and radiologic evidence of response to therapy can be reliably sought. The inventive molecular assay is useful prior to treatment to determine prospectively the potential level of responsiveness of a patient to EGFR modulators, taking a step forward in the development of individualized approaches to cancer therapy.

The invention is directed to evaluating c-fos dynamics to predict response to EGFR modulators in an in vitro and in vivo model. In addition, in vitro conditions may be reproducible to interrogate tumor material in an ex vivo manner. Strategies consisting in seriated biopsies are preferable to single, baseline evaluation to gain insight in the effects of therapy in a clinical setting.

Those skilled in the art will recognize, or be able to ascertain, using no more than routine experimentation, many equivalents to the specific embodiments of the invention described herein. Such equivalents are intended to be encompassed by the following claims.

All patents and references referred to herein are hereby incorporated by reference by their entirety.

1. A method for determining susceptibility of tumor cells in response to a chemotherapeutic agent prior to initiation of chemotherapy comprising the steps of:
introducing an Epidermal Growth Factor Receptor (EGFR) ligand or combination of ligand to a first volume of tumor cells,
introducing a Epidermal Growth Factor Receptor (EGFR) ligand or combination of ligand to a second and chemotherapeutic agent to second volume of tumor cells;
quantifying a level of c-fos mRNA in the first and second volumes of tumor cells; and
comparing the levels of c-fos mRNA to determine whether c-fos mRNA expression has been suppressed.

2. The method of claim 1, wherein introducing a chemotherapeutic agent comprises introducing an EGFR modulator.

3. The method of claim 1, wherein obtaining tumor cells comprises obtaining tumor cells via fine needle aspiration.

4. The method of claim 1 wherein introducing a chemotherapeutic agent comprises introducing a chemotherapeutic agent for from between about 1 minute and about 24 hours prior to the exposing EGFR ligand or combination of ligand to the second 1 volumes of tumor cells.

5. The method of claim 1 wherein the step of quantifying a level of c-fos mRNA is performed at predetermined time intervals between about 1 minute and 12 hours after the step of exposing EGFR ligand or combination of ligand to the second and third equal volumes of tumor cells.

6. The method of claim 1 wherein the steps of quantifying a level of c-fos mRNA is by quantitative real time polymerase chain reaction, rt-PCR, PCR, or qualitative real time PCR, following reverse transcription of mRNA into cDNA.

7. A molecular assay for measuring tumor response to therapy with EGFR modulator which utilizes tumor mRNA as a starting material comprising quantitative measurement of c-fos expression as an analytical endpoint.

8. The molecular assay of claim 7 further comprising incubating first, second, third and fourth volumes of tumor cells with media.

9. The molecular assay of claim 8 wherein the third and fourth equal volumes of tumor cells in media are exposed to a chemotherapeutic agent for a predetermined amount of time.

10. The molecular assay of claim 9 wherein an EGFR ligand or combination of ligand is exposed to the second and third equal volumes of tumor cells in media.

11. The molecular assay of claim 7 wherein the quantitative measurement of c-fos expression is determined by quantitative real time polymerase chain reaction, rt-PCR, PCR, or qualitative PCR, following reverse transcription of mRNA into cDNA.

12. A method for monitoring therapeutic effects of chemotherapeutic agents comprising:
providing tumor mRNA; and
measuring c-fos expression as an analytical endpoint.

13. The method of claim 12, wherein utilizing tumor mRNA as a starting material comprises extracting mRNA from tumor cells at predetermined intervals during treatment with an EGFR modulator.

14. The method of claim 13, wherein extracting mRNA from tumor cells at predetermined intervals during treatment with an EGFR modulator comprises extracting mRNA from tumor cells at predetermined intervals between one and fourteen days.

15. The method of claim 12, wherein extracting mRNA from tumor cells comprises the step of fine needle aspiration of tumor cells.

16. The method of claim 12, wherein measuring c-fos expression is determined by quantitative real time polymerase chain reaction.

17. A method for determining susceptibility of tumor cells in response to a chemotherapeutic agent prior to, during or after the initiation of chemotherapy comprising:
incubating first, second, third and fourth volumes of tumor cells with media;
introducing a chemotherapeutic agent to the third and fourth volumes of tumor cells in media;
exposing Epidermal Growth Factor Receptor (EGFR) ligand or combination of ligand to the second and third volumes of tumor cells in media;
quantifying a level of c-fos mRNA in the first, second, third and fourth volumes of tumor cells;
adjusting the levels of c-fos mRNA for comparison by dividing the c-fos mRNA levels in the first, second, third and fourth volumes of tumor cells by the c-fos mRNA level in the first volume of tumor cells; and
comparing the adjusted levels of c-fos mRNA for the second volume of tumor cells to the adjusted level of c-fos mRNA for the third equal volume of tumor cells to determine whether c-fos mRNA expression has been suppressed.

18. A method for determining susceptibility of tumor cells in response to a chemotherapeutic agent prior to initiation of chemotherapy comprising the steps of:
incubating first, second, third and fourth equal volumes of tumor cells with media;
introducing a chemotherapeutic agent to the third and fourth equal volumes of tumor cells in media;
exposing Epidermal Growth Factor Receptor (EGFR) ligand to the second and third equal volumes of tumor cells in media;
quantifying a level of c-fos mRNA in the first, second, third and fourth equal volumes of tumor cells;
adjusting the levels of c-fos mRNA for comparison by dividing the c-fos mRNA levels in the first, second, third and fourth equal volumes of tumor cells by the c-fos mRNA level in the fourth equal volume of tumor cells; and
comparing the adjusted levels of c-fos mRNA for the first equal volume of tumor cells to the adjusted level of c-fos mRNA for the third equal volume of tumor cells to determine whether c-fos mRNA expression has been suppressed.

19. The method of claim 18, wherein the comparing the adjusted levels of c-fos mRNA for the second equal volume of tumor cells to the adjusted level of c-fos mRNA for the third equal volume of tumor cells to determine whether c-fos mRNA expression has been suppressed.
